(19) United States
(12) Patent Application Publication
(10) Pub. No.: US 2023/0256091 A1
(43) Pub. Date: Aug. 17, 2023

(54) ANTI-TUMOR NANO ADJUVANT BASED ON VESICLE FORMED BY CROSS-LINKED BIODEGRADABLE POLYMER, PREPARATION METHOD THEREFOR AND USE THEREOF

(71) Applicant: SOOCHOW UNIVERSITY, Suzhou (CN)

(72) Inventors: Fenghua MENG, Suzhou (CN); Zhiyuan ZHONG, Suzhou (CN)

(21) Appl. No.: 18/012,218

(22) PCT Filed: Jan. 31, 2021
(86) PCT No.: PCT/CN2021/074571

§ 371 Date: Dec. 21, 2022

(30) Foreign Application Priority Data
Mar. 11, 2020 (CN) 202010167801.2

(51) Int. Cl. A61K 39/39; C08G 63/688; A61P 35/00

(52) U.S. Cl. CPC A61K 39/39; C08G 63/6882; A61P 35/00; A61K 2039/55561

(57) ABSTRACT

An anti-tumor nano adjuvant is obtained by loading a drug on the vesicle formed by a reversibly cross-linked biodegradable polymer with an asymmetric membrane structure; the drug is an oligonucleotide activating an immune response; the vesicle formed by the degradable polymer is obtained by the self-assembly of a polymer followed by cross-linking; the molecular chain of the polymer includes a hydrophilic chain segment, a hydrophobic chain segment and positively charged molecules, successively connected; the hydrophobic chain segment is a polycarbonate chain segment and/or a polyester chain segment, which is compounded and loaded with a drug by electrostatic interaction; and the membrane is a polycarbonate chain segment and/or a polyester chain segment, which is reversibly cross-linked, biodegradable and has good biocompatibility, the dithiolane in the side chain thereof is similar to thioctic acid, a natural antioxidant in human body, and the shell thereof is based on PEG and targets cancer cells.

### FIELD OF THE INVENTION

The present invention belongs to the drug carrier technology, and in particular relates to a preparation method for, and use of, an anti-tumor nano drug based on a vesicle formed by a cross-linked biodegradable polymer.

### BACKGROUND OF THE INVENTION

Glioblastoma (GBM) is a malignant brain cancer characterized by high recurrence, high metastatic rate, poor prognosis, and so on. At present, the standard clinical treatment usually includes surgical resection combined with chemotherapy and/or radiotherapy, but the therapeutic effect is not always satisfactory. In recent years, tumor immunotherapy has attracted extensive attention. However, due to the existence of the blood brain barrier (BBB), the immune adjuvant CpG cannot directly enter GBM; besides, the rapid degradation of CpG in vivo and the immunotoxicity caused by a high dose also limit its immunotherapy of CpG mainly through intratumoral/intracranial administration. Nevertheless, intracranial administration is usually accompanied by hydrocephalus, inflammation, and related toxic side effects caused by rapid diffusion of immune agonists into the blood, the CpG loading efficiency of the existing vesicle technology is low, and there are some problems such as unstable internal circulation of vesicles, low uptake of tumor cells, and low concentration of drugs in cells, which lead to low efficacy and toxic side effects of nano drugs, greatly limiting use of vesicles as carriers of such drugs.

### SUMMARY OF THE INVENTION

### Technical Problem

The purpose of the present invention is to disclose a preparation method for, and use of, an anti-tumor nano vaccine or nano adjuvant based on a vesicle formed by a cross-linked biodegradable polymer.

### Technical Solution

In order to achieve the above purpose, the present invention adopts the following technical solution:

An anti-tumor nano adjuvant based on a vesicle formed by a cross-linked biodegradable polymer is obtained by loading a drug on the vesicle formed by a reversibly cross-linked biodegradable polymer with an asymmetric membrane structure; the drug is an oligonucleotide that can activate an immune response; the vesicle formed by a reversibly cross-linked biodegradable polymer with an asymmetric membrane structure is obtained by means of the self-assembly of a polymer, or the self-assembly of a polymer and a targeting polymer; the polymer includes a hydrophilic chain segment, a hydrophobic chain segment and positively charged molecules; the targeting polymer includes a targeting molecule, a hydrophilic chain segment and a hydrophobic chain segment; and the hydrophobic chain segment is a polycarbonate chain segment and/or a polyester chain segment.

The present invention also discloses use of the vesicle formed by a reversibly cross-linked biodegradable polymer with an asymmetric membrane structure as a carrier of the oligonucleotide that can activate an immune response, or use of the vesicle in preparing a carrier of the oligonucleotide that can activate an immune response; the vesicle formed by a reversibly cross-linked biodegradable polymer with an asymmetric membrane structure is obtained by means of the self-assembly of a polymer, or the self-assembly of a polymer and a targeting polymer; the polymer includes a hydrophilic chain segment, a hydrophobic chain segment and positively charged molecules; the targeting polymer includes a targeting molecule, a hydrophilic chain segment and a hydrophobic chain segment; and the hydrophobic chain segment is a polycarbonate chain segment and/or a polyester chain segment.

In the present invention, the hydrophilic chain segment is polyethylene glycol; the hydrophobic chain segment contains a disulfide five-membered cyclic carbonate unit; the positively charged molecules include spermine and polyethyleneimine; and the molecular weight of the hydrophobic chain segment is 1.5-5 times, preferably 2-4 times, that of the hydrophilic chain segment, and the molecular weight of the positively charged molecule is 2%-40%, preferably 2.7%-24%, of that of the hydrophilic chain segment, for example, the hydrophilic chain segment is polyethylene glycol (Mₙ 5000-7500 Da), and the positively charged molecules are spermine (spermine, Mₙ 202) and polyethyleneimine (PEI, Mw 1200).

In the present invention, the chemical structural formula of the polymer is as follows:

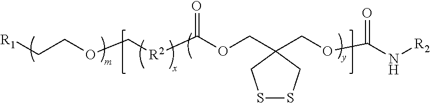

The chemical structural formula of the targeting polymer is as follows:

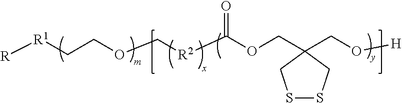

Where R₁ is an end group of the hydrophilic chain segment; R₂ is a positively charged molecule; R is a targeting molecule; R¹ is a targeting molecule linkage group; and R² is an ester unit or a carbonate unit, i.e. a cyclic ester monomer or a unit of a cyclic carbonate monomer after ring opening.

Preferably, the molecular weight of PEG is 5000-7500 Da; the total molecular weight of the R² chain segment is 2.5-4 times that of PEG; the total molecular weight of PDTC is 10%-30% of that of the R² chain segment; the molecular weight of PEI is 7%-24% of that of PEG; and the molecular weight of spermine is 2.7%-4% of that of PEG.

Further, the disulfide five-membered cyclic unit is obtained by ring opening of the cyclic carbonate monomer (DTC) containing a disulfide five-membered cyclic functional group.

For example, the chemical structural formula of the polymer in the present invention is as follows:

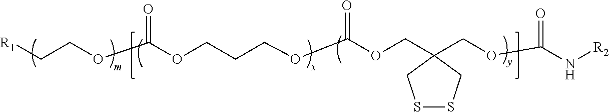

The chemical structural formula of the targeting polymer is as follows:

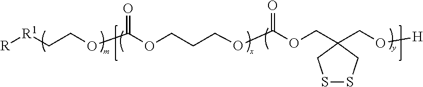

Preferably, the molecular weight of PEG is 5000-7500 Da; the total molecular weight of PTMC is 2.5-4 times that of PEG; the total molecular weight of PDTC is 10%-30% of th at of PTMC; the molecular weight of PEI is 7%-24% of that of PEG; and the molecular weight of spermine is 2.7%-4% of that of PEG.

In the present invention, the oligonucleotide that can activate an immune response is a CpG drug, such as CpG ODN 1826, CpG ODN 2395 and CpG ODN 2006, with the specific sequence belonging to the prior art.

In the polymer of the present invention, when small molecule spermine and low molecular weight branched PEI (PEI1.2k) with good biocompatibility are used as carriers, the toxicity is low; and when a PEG chain segment and a hydrophobic chain segment are introduced by combination, a good drug entrapment rate can be achieved, so that even when the content of the drug is up to 15 wt. %, the vesicle can still completely encapsulate the drug; in addition, the polymer of the present invention avoids the defects of instability caused by existing PEI combining drugs through physical winding, being positively charged, and weak migration due to easy combination with cells, combines drugs by electrostatic force, and is then separated from the outside by the cross-linked vesicle membrane, so as to avoid losses and toxic side effects caused by cell adhesion in the transport process, and it can efficiently migrate to a nidus by modification of specific targeting molecules.

For the vesicle formed by a reduction sensitive reversibly cross-linked, intracellular de-crosslinkable biodegradable polymer with an asymmetric membrane structure designed in the present invention, the outer surface of the vesicle membrane is composed of non-adhesive polyethylene glycol (PEG) and is preferably modified with the targeting molecule ApoE polypeptide, and the inner surface of the vesicle membrane is composed of small molecule spermine and low molecular weight branched PEI (PEI1.2k) with good biocompatibility and is used to efficiently load the oligonucleotide CpG that can activate an immune response; the cross-linked vesicular membrane can protect the drug from degradation and leakage, and can circulate in vivo for a long time; and the nano size of the vesicle and the tumor-specific targeting molecules on the surface enable the vesicle to deliver drugs into tumor cells directionally through veins or nasal veins.

In the polymer or targeting polymer of the present invention, the R² chain segment of the middle block and DTC are arranged randomly; spermine and PEI, smaller than PEG in the molecular weight, can be used to obtain a vesicle formed by a cross-linked polymer with an asymmetric membrane structure after self-assembling and cross-linking, the inner shell of the vesicle membrane being positively charged spermine or PEI and being used for compounding the drug CpG; and the vesicle membrane is P(R²-DTC), which is reversibly cross-linked, biodegradable and has good biocompatibility; and the dithiolane in the side chain thereof is similar to thioctic acid, a natural antioxidant in the human body, and can provide reduction sensitive reversible cross-linking and support the long circulation of biodrugs in the blood.

The present invention also discloses a preparation method for the anti-tumor nano adjuvant based on a vesicle formed by a cross-linked biodegradable polymer, which comprises the following steps: preparing the anti-tumor nano adjuvant based on a vesicle formed by a cross-linked biodegradable polymer by a solvent displacement method using a polymer and an oligonucleotide that can activate an immune response as raw materials; or preparing the anti-tumor nano adjuvant based on a vesicle formed by a cross-linked biodegradable polymer by a solvent displacement method using a polymer, a targeting polymer, and an oligonucleotide that can activate an immune response as raw materials.

In the present invention, the targeting molecule is ApoE polypeptide (sequence: LRKLRKRLLLRKLRKRLLC); MeO-PEG-P(R²-DTC)-SP or PEG-P(R²-DTC)-PEI1.2k is mixed with a diblock polymer (e.g. ApoE-PEG-P(R²-DTC)) coupled with an active tumor-targeting molecule, and after co-self-assembling, drug loading and cross-linking, an active tumor-targeting anti-tumor drug with an asymmetric membrane structure is obtained.

The present invention discloses use of the above anti-tumor nano vaccine based on a vesicle formed by a cross-linked biodegradable polymer in preparing anti-tumor drugs, preferably in preparing anti-brain glioma drugs.

It is common knowledge that the way of administration is one of the key factors in the treatment of tumors, especially for tumors of the brain, which is different from other tissues. The treatment of brain glioma with CpG of the prior art is mostly carried out through intracranial administration, which is determined by the inherent nature of CpG, because CpG has strong water solubility and, as a small molecule immune adjuvant, needs to enter the antigen presenting cell APC to play its role. Therefore, CpG requires intratumoral administration to be close to the APC already infiltrated in the tumor, so that it can enter the APC. Despite this way of administration, the existing technology still cannot solve the problem that CpG, having small molecules, may quickly spread into the blood even through intratumoral administration, leading to systemic immunotoxicity. Moreover, for a brain tumor in situ, the drug administration within the tumor, i.e. the brain, will cause great damage, usually accompanied by hydrocephalus and easy infection. The present invention creatively provides an anti-tumor nano adjuvant based on a vesicle formed by a cross-linked biodegradable polymer, and thus solves the problem that CpG is highly water-soluble, negatively charged and difficult to enter APC; in particular, the drug of the present invention can be effectively administered by intravenous injection, such as caudal vein injection, so that the technical prejudice of the prior art that only intracranial administration can be used is overcome, not only achieving an excellent therapeutic effect, but also solving the defects existing in the existing administration methods.

### Advantageous Effects of the Invention

The present invention has the following advantages compared to the prior art: 1. The vesicle formed by a cross-linked polymer with an asymmetric membrane structure in the anti-tumor nano adjuvant based on a vesicle formed by a cross-linked biodegradable polymer disclosed by the present invention are used for in-vivo transmission; the inner shell of the vesicle membrane being spermine SP or PEI and being used for compounding the nucleic acid drug CpG; the vesicle membrane is PTMC, which is reversibly cross-linked, biodegradable and has good biocompatibility; the dithiolane in the side chain thereof is similar to thioctic acid, a natural antioxidant in the human body, and can provide reduction sensitive reversible cross-linking and support the long circulation of nano drugs in the blood; and the shell thereof is based on PEG, can have targeting molecules, and can bind to cancer cells with high specificity.

2. The anti-tumor drug disclosed by the present invention, loading the nucleic acid drug CpG on the vesicle formed by a cross-linked polymer with an asymmetric membrane structure, was applied to in-vivo treatment of in-situ mouse brain glioma LCPN model mice, with the results indicating that the vesicle loaded with a drug has many unique advantages, including simple manipulation of preparation, excellent biocompatibility, superior targeting to cancer cells, and significant ability to inhibit weight loss and prolong the survival period. Therefore, the vesicle system of the present invention is expected to become a nano-system platform integrating advantages such as being convenient and fast, targeting, and multifunctional, so as to be used for efficient and active targeting delivery of nucleic acid and other drugs to tumors, including in-situ brain tumors.

3. For the vesicle formed by a reduction sensitive reversibly cross-linked, intracellular de-crosslinkable biodegradable polymer with an asymmetric membrane structure in the anti-tumor drug disclosed by the present invention, the outer surface of the vesicle membrane is composed of non-adhesive polyethylene glycol (PEG) and is modified with ApoE polypeptide that can specifically target LDLRs, and the inner surface of the vesicle membrane is composed of small molecule spermine and low molecular weight branched PEI (PEI1.2k) with good biocompatibility and is used to efficiently load the oligonucleotide CpG that can activate an immune response; the cross-linked vesicular membrane can protect the drug from degradation and leakage, and can circulate in vivo for a long time; and the nano size of the vesicle and the tumor-specific targeting molecules on the surface enable the vesicle to deliver drugs into tumor cells directionally through veins or nasal veins.

4. The vesicle formed by a polymer with an asymmetric membrane structure in the anti-tumor drug disclosed by the present invention is a cross-linked vesicle, and spermine or PEI cooperates with a hydrophilic chain segment and a hydrophobic chain segment, so that the vesicle has stable structure and good circulation in vivo; the vesicle can completely encapsulate up to 15 wt. % of the drug, indicating that the anti-tumor drug of the present invention has excellent stability; the vesicle, after the outer surface of the membrane thereof is modified with ApoE polypeptide that can specifically target LDLRs, can have significant enrichment and therapeutic effects at the in-situ brain glioma site by administration through veins or nasal veins, is a good controlled-release carrier for nucleic acid drugs, and can be used as a separate nano vaccine or nano immune adjuvant for efficient immunotherapy of tumors.

### BRIEF DESCRIPTION OF THE DRAWINGS

FIG. 1 is the nuclear magnetic map of PEG5k-P(TMC14.9k-DTC2.0k) in Example 1.

FIG. 2 is the nuclear magnetic map of Mal-PEG7.5k-P(TMC15.2k-DTC2.0k) in Example 2.

FIG. 3 is the nuclear magnetic map of PEG5k-P(TMC14.9k-DTC2.0k)-b-spermine in Example 3.

FIG. 4 is the nuclear magnetic map of PEG5k-P(TMC14.9k-DTC2.0k)-b-PEI1.2k in Example 4.

FIG. 5 is the nuclear magnetic map of ApoE-PEG7.5k-P(TMC15.2k-DTC2.0k) in Example 5.

FIG. 6 shows the particle size distribution of the targeting drug-loaded vesicle ApoE-PS-CpG in Example 6.

FIG. 7 is the flow endocytosis diagram of the vesicles ApoE-PS with different targeting densities for LCPN cells in Example 8.

FIG. 8 shows the therapeutic effects of different CpG formulations and different dosages on in-situ mouse brain glioma LCPN model mice studied by caudal vein administration in Example 9.

FIG. 9 shows the therapeutic effects of ApoE-PS-Sp-CpG combined with radiotherapy on in-situ mouse brain glioma LCPN model mice studied by caudal vein administration in Example 10.

FIG. 10 shows the therapeutic effects of ApoE-PS-Sp-CpG combined with αCTLA-4 on in-situ mouse brain glioma LCPN model mice studied by caudal vein administration in Example 11.

FIG. 11 shows the therapeutic effects of ApoE-PS-PEI1.2k-CpG and ApoE-PS-Sp-CpG on in-situ mouse brain glioma LCPN model mice compared by caudal vein administration in Example 12.

FIG. 12 shows the therapeutic effects of different CpG formulations on in-situ mouse brain glioma LCPN model mice studied by nasal vein administration.

FIG. 13 shows the therapeutic effects of ApoE-PS-PEI1.2k-CpG combined with radiotherapy on in-situ mouse brain glioma LCPN model mice studied by nasal vein administration.

FIG. 14 shows the analysis of immune cells in the tumor and spleen of mice bearing in-situ LCPN.

FIG. 15 shows the effects of in-vitro simulation of different CpG formulations penetrating BBB.

FIG. 16 shows the effects of different empty carriers and CpG formulations on activating BMDC in vitro.

FIG. 17 shows the in-vivo pharmacokinetics of different CpG formulations and the biological distribution of main organs.

FIG. 18 shows the effects of different CpG formulations on activating immune cells in tumors and lymph nodes.

### INVENTION EMBODIMENTS

### Embodiments of the Invention

The present invention will be further described below with reference to examples and drawings. In the present invention, the chemical structural formula of the polymer is as follows:

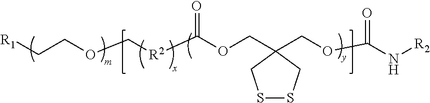

The chemical structural formula of the targeting polymer is as follows:

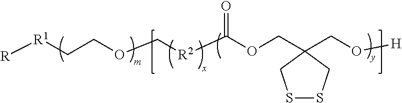

Where R₁ is an end group of the hydrophilic chain segment; R₂ is a positively charged molecule; R is a targeting molecule; and R¹ is a targeting molecular linkage group.

R² is a cyclic ester monomer, or a unit of a cyclic carbonate monomer after ring opening, for example, the cyclic ester monomer includes caprolactone (8-CL), lactide (LA) or glycolide (GA), and the cyclic carbonate monomer includes trimethylene cyclic carbonate (TMC); preferably, when R² is TMC, the chemical structural formula of the polymer is as follows:

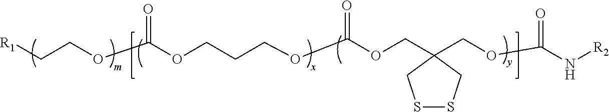

Where R₂ is a positively charged molecule; R₁ is an end group of the hydrophilic chain segment, such as

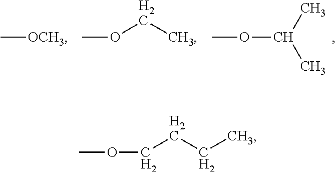

The targeting polymer is obtained by the conventional reaction of the polymer B and the targeting molecule at the R¹¹ group, the R¹¹ group corresponding to the R¹ group after the reaction.

The chemical structural formula of the polymer B is as follows:

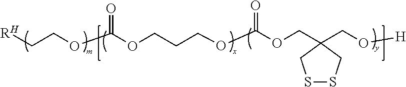

Where R¹¹ is a targeting molecular linkage group, such as

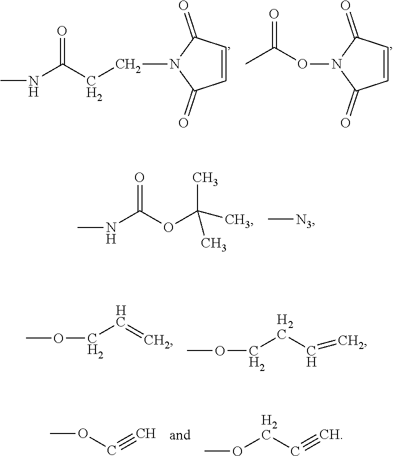

As a preferred example, the present invention uses methoxy terminated PEG and Mal groups as the linkage groups (R₁ and R¹¹, respectively):

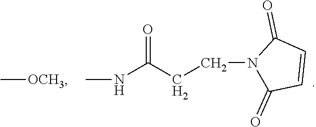

R₂ is selected from one of the following groups:

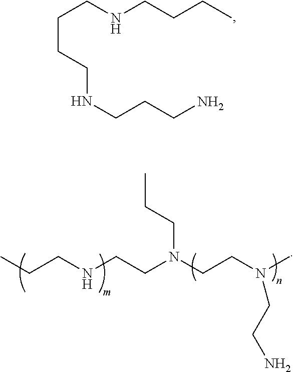

As a preferred example, the preparation method for the polymer and targeting polymer of the present invention is as follows: activating the terminal hydroxyl group of MeO-PEG-P(TMC-DTC)-OH by a hydroxyl activator N,N′-carbonyl diimidazole (CDI), and then reacting with spermine or PEI to obtain MeO-PEG-P(TMC-DTC)-Sp or MeO-PEG-P(TMC-DTC)-PEI; and, at the Mal end of PEG of Mal-PEG-P(TMC-DTC), coupling the tumor-specific targeting molecule (ApoE polypeptide) through the Michael addition reaction to obtain the targeting ApoE-PEG-P(TMC-DTC).

As a preferred example, the preparation method for the anti-tumor nano adjuvant based on a vesicle formed by a cross-linked biodegradable polymer of the present invention is as follows: preparing the anti-tumor nano adjuvant based on a vesicle formed by a cross-linked biodegradable polymer by a solvent displacement method using MeO-PEG-P(TMC-DTC)-Sp and a drug as raw materials; or preparing the anti-tumor nano drug based on a vesicle formed by a cross-linked biodegradable polymer by a solvent displacement method using MeO-PEG-P(TMC-DTC)-PEI and a drug as raw materials; or preparing the anti-tumor nano drug based on a vesicle formed by a cross-linked biodegradable polymer by a solvent displacement method using MeO-PEG-P(TMC-DTC)-Sp, ApoE-PEG-P(TMC-DTC) and a drug as raw materials; or preparing the anti-tumor nano drug based on a vesicle formed by a cross-linked biodegradable polymer by a solvent displacement method using MeO-PEG-P(TMC-DTC)-PEI, ApoE-PEG-P(TMC-DTC) and a drug as raw materials.

The above preparation method specifically comprises the following steps: making MeO-PEG-P(TMC-DTC)-OH and a hydroxyl activator react in a dry solvent, and then precipitating, suction-filtering, and vacuum-drying to obtain MeO-PEG-P(TMC-DTC)-CDI with an activated terminal hydroxyl group; dropping its solution into spermine or a PEI solution for reaction, and then precipitating, suction-filtering, and vacuum-drying to obtain MeO-PEG-P(TMC-DTC)-Sp or MeO-PEG-P(TMC-DTC)-PEI.

Making Mal-PEG-P(TMC-DTC) react with ApoE polypeptide dissolved in an organic solvent to obtain the targeting ApoE-PEG-P(TMC-DTC).

Adding the raw material solution to a non-ionic buffer solution, placing at room temperature, and then dialyzing and cross-linking to obtain an anti-tumor nano drug based on a vesicle formed by a cross-linked biodegradable polymer.

All the raw materials involved in the examples of the present invention are existing products, such as PEG, Mal-PEG, TMC, DTC, DPP, and the oligonucleotide CpG that can activate an immune response; the LCPN cells are mouse malignant brain glioma cells from Institute of FUNSOM, Soochow University, and the obtained in-situ mouse model can better reflect the effects of drugs, especially the immune effect, compared with the mouse model of heterotransplanted human brain glioma.

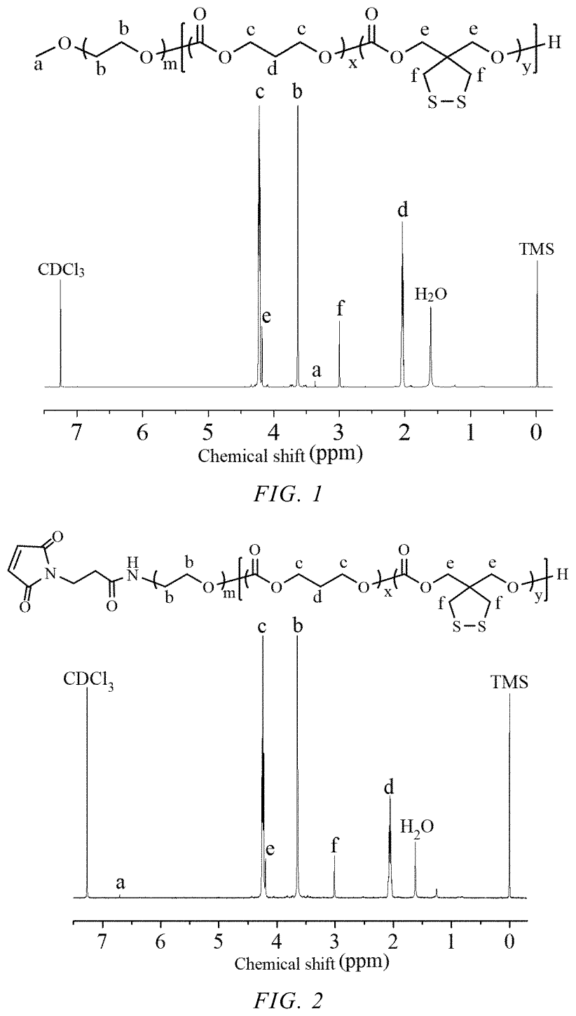

Example 1: Synthesis of MeO-PEG5k-P(TMC14.9k-DTC2.0k) block copolymer:MeO-PEG5k-P(TMC14.9k-DTC2.0k) was prepared by ring opening polymerization, which was specifically as follows: in a nitrogen glovebox, weighing MeO-PEG-OH (Mₙ=5.0 kg/mol, 0.50 g, 100 μmol), TMC (1.5 g, 14.7 mmol), DTC (0.2 g, 1.0 mmol) and diphenyl phosphate (DPP, 0.25 g, 1000 μmol) in sequence, and dissolving them in dichloromethane (DCM, 7.9 mL); sealing in a closed reactor, and then putting the reactor in an oil bath at 40° C. to react for 3 days under magnetic stirring; and then precipitating twice in ice ether, suction-filtering, and vacuum-drying at room temperature to obtain a product at a yield of about 90%. ¹H NMR (400 MHz, CDCl₃): PEG: d 3.38, 3.65; TMC: d 4.24, 2.05; DTC: d 4.32, 3.02. FIG. 1 showed the nuclear magnetic spectrum of MeO-PEG5k-P(TMC14.9k-DTC2.0k); it could be known from the integration that the molecular weight of the final polymer was PEG5k-P(TMC14.9k-DTC2.0k):

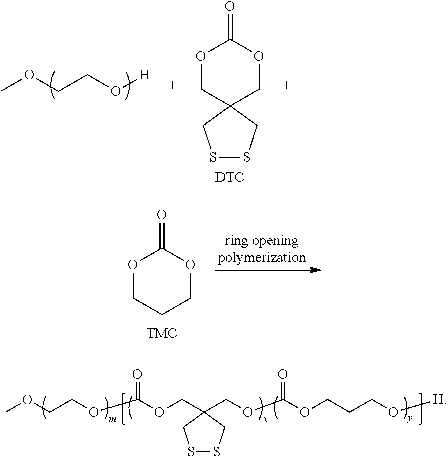

PEG5k-P(CL15.9k-DTC2.0k) was obtained when the above TMC was replaced with caprolactone, with the molar weight and other conditions remained unchanged:

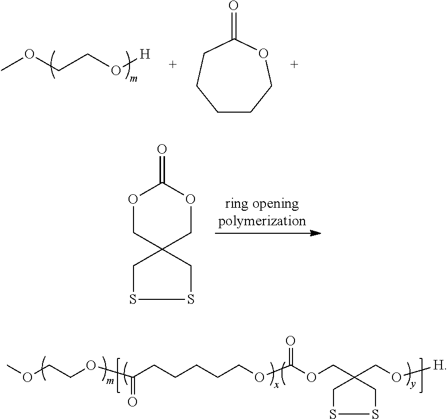

PEG5k-P(TMBPEC10.3k-DTC2.0k) was obtained when the above TMC was replaced with a 2,4,6-trimethoxy phenyl acetal pentaerythritol carbonate (TMBPEC) monomer, with the molar weight and other conditions remained unchanged:

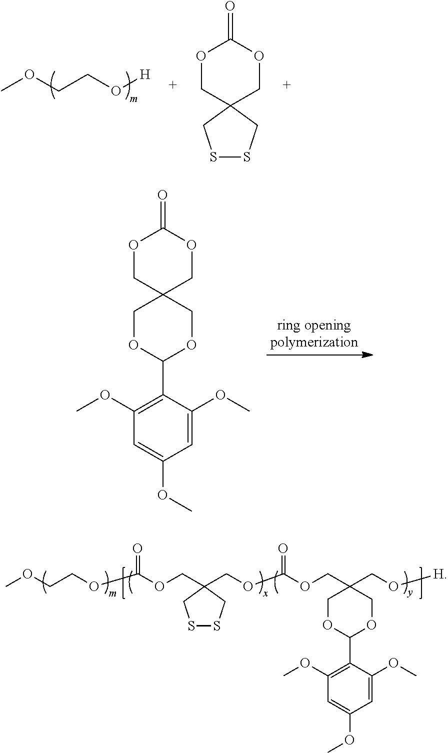

PEG5k-P(LA13.1k-DTC1.9k) was obtained when the above TMC was replaced with lactide and the catalyst was replaced with 1,8-diazabicycloundecen-7-ene DBU (50 μmol), DCM 28 mL, and the reaction was carried out at 30° C. for 3 h, with the molar weight of other substances and other conditions remained unchanged:

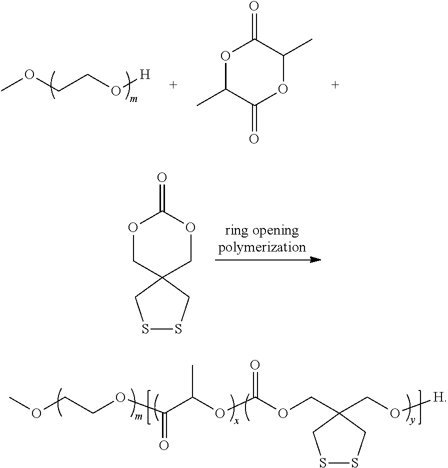

PEG5k-P(GA10.1k-DTC1.8k) was obtained when the above TMC was replaced with glycolide and the catalyst was replaced with 1,8-diazabicycloundecen-7-ene DBU (50 μmol), DCM 28 mL, and the reaction was carried out at 30° C. for 3 h, with the molar weight of other substances and other conditions remained unchanged.

Example 2: Synthesis of Mal-PEG7.5k-P(TMC15.2k-DTC2.0k) block copolymer: The Mal-PEG7.5k-P(TMC15.2k-DTC2.0k) block copolymer was prepared by ring opening polymerization, which was specifically as follows: in a nitrogen glovebox, weighing Mal-PEG-OH (Mₙ=7.5 kg/mol, 0.75 g, 100 μmol), TMC (1.5 g, 14.7 mmol), DTC (0.2 g, 1.0 mmol) and diphenyl phosphate (DPP, 0.25 g, 1000 μmol) in sequence, and dissolving them in dichloromethane (DCM, 7.9 mL); sealing in a closed reactor, and then putting the reactor in an oil bath at 40° C. to react for 3 days under magnetic stirring; and then precipitating twice in ice ether, suction-filtering, and vacuum-drying at room temperature to obtain a product at a yield of about 90%. ¹H NMR (400 MHz, CDCl₃): PEG: d 3.38, 3.65; TMC: d 4.24, 2.05; DTC: d 4.32, 3.02; Mal: d 6.8. FIG. 2 showed the nuclear magnetic spectrum of Mal-PEG7.5k-P(TMC15.2k-DTC2.0k); it could be known from the integration that the molecular weight of the final polymer was Mal-PEG7.5k-P(TMC15.2k-DTC2.0k).

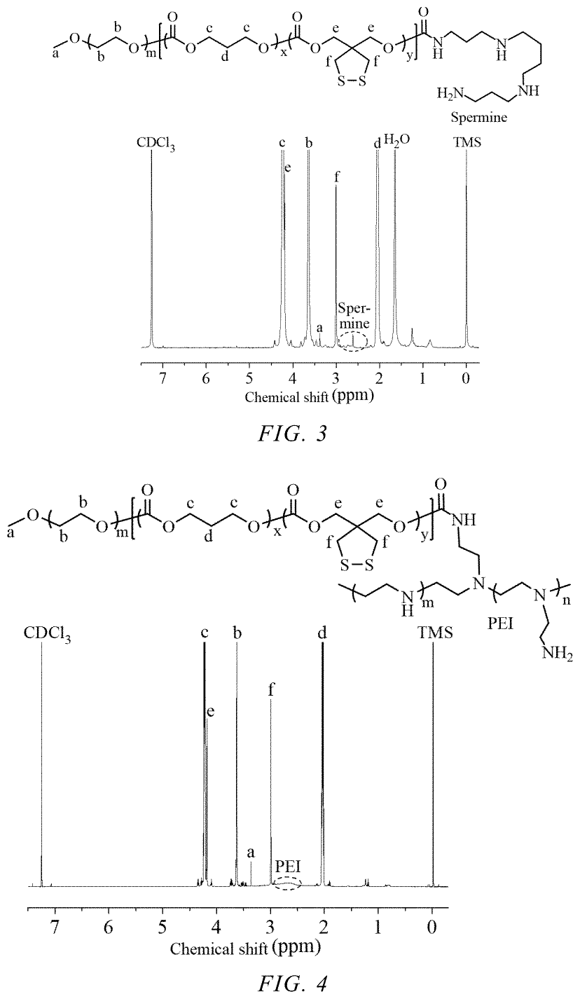

Example 3: Synthesis of PEG5k-P(TMC14.9k-DTC2.0k)-Sp block copolymer: The synthesis of PEG5k-P(TMC14.9k-DTC2.0k)-Sp was divided into two steps; with all the reactions carried out under the anhydrous and oxygen free conditions, first the terminal hydroxyl group of PEG5k-P(TMC14.9k-DTC2.0k) was activated with N,N′-carbonyl diimidazole (CDI), and then PEG5k-P(TMC14.9k-DTC2.0k) was made to react with the primary amine of spermine. Specifically, first dissolving PEG5k-P(TMC14.9k-DTC2.0k) (2.2 g, hydroxyl 0.1 mmol) and CDI (48.6 mg, 0.3 mmol) in 11 mL of dry DCM and reacting at 30° C. for 4 h, and then precipitating twice in ice ether, filtering, and vacuum-drying to obtain PEG5k-P(TMC14.9k-DTC2.0k)-CDI; then weighing 1.6 g of the product (0.07 mmol) from the previous step, and dissolving it in 8 mL of DCM; then, under the stirring condition in an ice water bath, adding the obtained solution to 7 mL of DMSO containing spermine (141.4 mg, 0.7 mmol) drop by drop through a constant pressure dropping funnel for about 2 h; then transferring to 30° C. and continuing to react for 4 h; and then precipitating twice in ice ethanol, suction-filtering, and vacuum-drying at room temperature to obtain PEG5k-P(TMC14.9k-DTC2.0k)-Sp at a yield of about 90%. ¹H NMR (400 MHz, CDCl₃): PEG: d 3.38, 3.65; TMC: d 4.24, 2.05; DTC: d 4.32, 3.02; spermine: d 2.6-2.8; ¹H NMR characterization showed that in addition to PEG and P(DTC-TMC) peaks, there were also characteristic peaks of spermine at d 2.6-2.8. FIG. 3 showed the nuclear magnetic spectrum of PEG5k-P(TMC14.9k-DTC2.0k)-Sp; it could be known from the integration that the grafting rate of spermine was above 90%.

With TMC replaced, PEG5k-P(CL15.9k-DTC2.0k)-Sp, PEG5k-P(TMBPEC10.3k-DTC2.0k)-Sp, PEG5k-P(LA13.1k-DTC1.9k)-Sp and PEG5k-P(GA10.1k-DTC1.8k)-Sp could be prepared according to the above method; and it could be known from the nuclear magnetic integral that the grafting rate of spermine was above 90%.

Example 4: Synthesis of PEG5k-P(TMC14.9k-DTC2.0k)-PEI1.2k block copolymer: The synthesis of PEG5k-P(TMC14.9k-DTC2.0k)-PEI1.2k was divided into two steps; with all the reactions carried out under the anhydrous and oxygen free conditions, first the terminal hydroxyl group of PEG5k-P(TMC14.9k-DTC2.0k) was activated with N,N′-carbonyl diimidazole (CDI), and then PEG5k-P(TMC14.9k-DTC2.0k) was made to react with the primary amine of PEI1.2k. The steps were specifically as follows: first dissolving PEG5k-P(TMC14.9k-DTC2.0k) (2.2 g, hydroxyl 0.1 mmol) and CDI (48.6 mg, 0.3 mmol) in 11 mL of dry DCM and reacting at 30° C. for 4 h, and then precipitating twice in ice ether, filtering, and vacuum-drying to obtain PEG5k-P(TMC14.9k-DTC2.0k)-CDI; then weighing 1.6 g of the product (0.07 mmol) from the previous step, and dissolving it in 8 mL of DCM; then, under the stirring condition in an ice water bath, adding the obtained solution to 17 mL of DCM containing PEI1.2k (840 mg, 0.7 mmol) drop by drop through a constant pressure dropping funnel for about 2 h; then transferring to 30° C. and continuing to react for 4 h; and then precipitating for three times in ice ethanol/ice ether (v/v, 1/3), suction-filtering, and vacuum-drying at room temperature to obtain the product at a yield of about 70%. ¹H NMR (400 MHz, CDCl₃): PEG: d 3.38, 3.65; TMC: d 4.24, 2.05; DTC: d 4.32, 3.02; PEI1.2k: d 2.5-2.8; ¹H NMR characterization showed that in addition to PEG and P(DTC-TMC) peaks, there were also characteristic peaks of PEI1.2k at d 2.5-2.8. FIG. 4 showed the nuclear magnetic spectrum of PEG5k-P(TMC14.9k-DTC2.0k)-PEI1.2k; it could be known from the integration that the grafting rate of PEI1.2k was above 90%.

With TMC replaced, PEG5k-P(CL15.9k-DTC2.0k)-PEI1.2, PEG5k-P(TMBPEC10.3k-DTC2.0k)-PEI1.2, PEG5k-P(LA13.1k-DTC1.9k)-PEI1.2 and PEG5k-P(GA10.1k-DTC1.8k)-PEI1.2k could be prepared according to the above method; and it could be known from the nuclear magnetic integral that the grafting rate of PEI was above 90%.

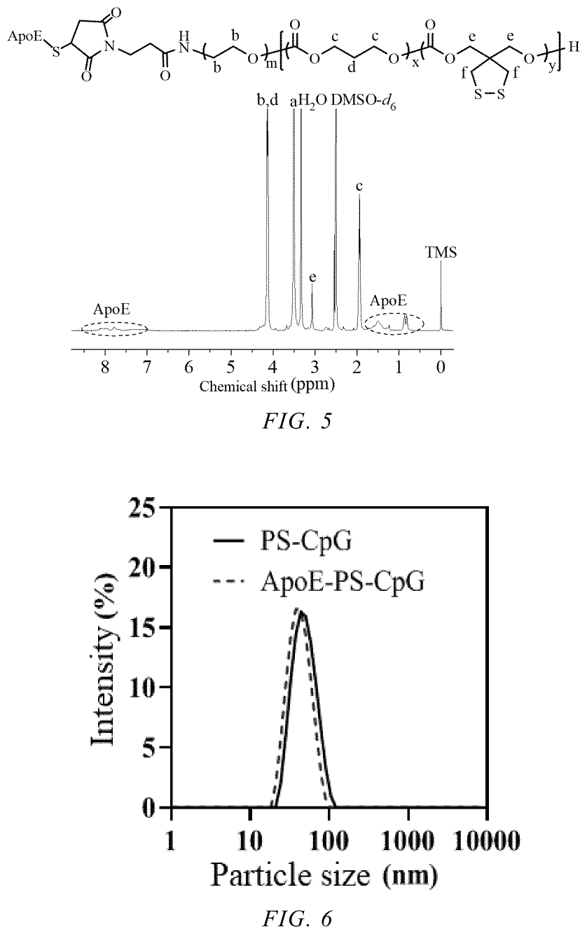

Example 5: Synthesis of targeting diblock copolymer ApoE-PEG7.5k-P(TMC15.2k-DTC2.0k): The synthesis of ApoE-PEG7.5k-P(TMC15.2k-DTC2.0k) was realized by bonding the polypeptide ApoE-SH with a free thiol group to Mal-PEG7.5k-P(TMC15.2k-DTC2.0k) through the Michael reaction. The steps were briefly as follows: under the protection of nitrogen, dissolving Mal-PEG7.5k-P(TMC15.2k-DTC2.0k) (247 mg, 0.01 mmol) and ApoE-SH (30 mg, 0.012 mmol) successively in 2.5 mL of DMF, and then reacting at 37° C. for 8 h; then, at room temperature, dialyzing the reactants with DMSO (MWCO 7000 Da) for 6 h (with the dialysate changed for three times), and then dialyzing with DCM for 6 h (with the dialysis medium changed for three times); and then precipitating twice in ice ethanol, suction-filtering, and vacuum-drying at room temperature to obtain the product at a yield of 85%. FIG. 5 showed the nuclear magnetic spectrum of ApoE-PEG7.5k-P(TMC15.2k-DTC2.0k), which indicated that in addition to PEG and P(DTC-TMC) peaks, there were also characteristic peaks of ApoE at d 0.8-1.8 and 4.2-8.2. A BCA protein analysis kit was used to establish a standard curve at 492 nm with an ApoE sample of known concentration, and then the grafting ratio of ApoE could be determined. After analysis, the grafting ratio of ApoE of the targeting polymer was 95%.

With TMC replaced, ApoE-PEG7.5k-P(CL15.6k-DTC1.9k), ApoE-PEG7.5k-P(LA11.8k-DTC1.7k), ApoE-PEG7.5k-P(GA9.8k-DTC1.6k) and ApoE-PEG7.5k-P(TMBPEC10.0k-DTC1.9k) could be prepared according to the above method; and the grafting ratio of ApoE of the targeting polymer was 90%-95%.

It was verified by nuclear magnetic testing that the above products were the designed products; and the above polymers and targeting polymers were used to prepare a drug-loaded vesicle in the following examples.

Example 6: Preparation of targeting drug-loaded vesicle based on PEG5k-P(TMC14.9k-DTC2.0k)-Sp: ApoE-PS-Sp-CpG with different ApoE targeting densities loaded with CpG was prepared by a solvent exchange method. The specific steps were as follows: adding a certain amount of CpG (CpG ODN 1826, with a theoretical drug-loading rate of 10 wt. %) to 950 μL of a HEPES buffer solution (5 mM, pH 6.8), then adding 50 μL of a DMSO solution of ApoE-PEG-P(TMC-DTC) and MeO-PEG-P(TMC-DTC)-SP (at a molar ratio of 1:4 and a total polymer concentration of 40 mg/mL) to HEPES and stirring for 10 min, and then dialyzing the obtained vesicles in HEPES for 2 h (MWCO 350 kDa), in the mixed liquid of HEPES and a PB buffer solution (10 mM, pH 7.4) (v/v, 1/1) for 1 h, and in PB for 2 h to obtain a targeting drug-loaded vesicle, which was recorded as ApoE-PS-Sp-CpG, a 20% ApoE targeting group. The drug-loading rate and entrapment rate of CpG were determined with Nanodrop. The results showed that when the theoretical drug-loading rate was 10 wt. %, the entrapment rate was 100%, that is, the theoretical drug-loading rate was consistent with the actual drug-loading rate. FIG. 6 showed a particle size distribution diagram of the above vesicle, indicating that the particle size was about 50 nm and the particle size distribution was narrow.

When TMC was replaced respectively with caprolactone (8-CL), lactide (LA), glycolide (GA), and a 2,4,6-trimethoxy phenyl acetal pentaerythritol carbonate (TMBPEC) monomer, a targeting drug-loaded cross-linked vesicle loaded with CpG was obtained according to the above method with an entrapment rate of 96%, 83%, 92% and 85%, respectively.

When CpG ODN 1826 was replaced with CpG ODN 2395 or CpG ODN 2006 and the rest remained unchanged, ApoE targeting drug-loaded cross-linked vesicles were obtained according to the above method with an entrapment rate of 100%.

When the above theoretical drug-loading rate was changed to 5 wt. % and the rest remained unchanged, an ApoE targeting drug-loaded cross-linked vesicle was obtained. When the theoretical drug-loading rate was 5 wt. % as determined by Nanodrop, the entrapment rate of CpG was 100%, that is, the theoretical drug-loading rate was consistent with the actual drug-loading rate; and the particle size of the vesicles obtained was about 50 nm with a narrow distribution.

When the molar ratio of ApoE-PEG-P(TMC-DTC) and MeO-PEG-P(TMC-DTC)-SP was changed and the rest remained unchanged, the drug-loaded cross-linked vesicles with different ApoE targeting densities (5% ApoE targeting group, 10% ApoE targeting group, 15% ApoE targeting group, 25% ApoE targeting group, 30% ApoE targeting group, and 35% ApoE targeting group) were obtained. The drug-loading rate and entrapment rate of CpG were determined with Nanodrop. The results showed that the theoretical drug-loading rate was 5 wt. %, and the entrapment rate of the targeting drug-loaded vesicles was close to 100%; when the theoretical drug-loading rate was 10 wt. %, the entrapment rate of each targeting group was 100%, 100%, 100%, 95%, 90% and 84%, respectively. The particle size of all the vesicles was 50-80 nm with a narrow distribution.

PS-Sp-CpG loaded with CpG was prepared by a solvent exchange method. The specific steps were as follows: adding a certain amount of CpG (with a theoretical drug-loading rate of 5 wt. % and 10 wt. %, respectively) to 950 μL of a HEPES buffer solution (5 mM, pH 6.8), and then adding 50 μL of a DMSO solution of MeO-PEG-P(TMC-DTC)-SP (at a polymer concentration of 40 mg/mL) to a HEPES buffer solution and stirring for 10 min; and dialyzing the obtained dispersion in the HEPES buffer solution for 2 h (MWCO 350 kDa), in a mixed buffer solution of HEPES and PB (10 mM, pH 7.4) (v/v, 1/1) for 1 h, and in a PB buffer solution for 2 h to obtain a targeting drug-loaded vesicle, which was recorded as PS-Sp-CpG (with a drug-loading rate of 10 wt. %). The drug-loading rate and entrapment rate of CpG were determined with Nanodrop. The results showed that when the theoretical drug-loading rate was 5 wt. % and 10 wt. %, the entrapment rate was 100%, that is, the theoretical drug-loading rate was consistent with the actual drug-loading rate. The particle size of the vesicles obtained above was 50-55 nm with a narrow distribution.

Example 7: Preparation of targeting drug-loaded vesicle based on PEG5k-P(TMC14.9k-DTC2.0k)-PEI1.2k: ApoE-PS-PEI-CpG with different ApoE targeting densities loaded with CpG was prepared by a solvent exchange method. The specific steps were as follows: adding a certain amount of CpG (with a theoretical drug-loading rate of 10 wt. %) to 950 μL of a HEPES buffer solution (5 mM, pH 6.8), and then adding 50 μL of a DMSO solution of ApoE-PEG-P(TMC-DTC) and MeO-PEG-P(TMC-DTC)-PEI1.2k (at a molar ratio of 1:9 and a total polymer concentration of 40 mg/mL) to HEPES and stirring for about 10 min; and dialyzing the obtained vesicles in HEPES for 2 h (MWCO 350 kDa), in a mixed buffer solution of HEPES and PB (10 mM, pH 7.4) (v/v, 1/1) for 1 h, and in a PB buffer solution for 2 h to obtain a targeting drug-loaded vesicle, which was recorded as ApoE-PS-PEI-CpG, a 10% ApoE targeting group. The drug-loading rate and entrapment rate of CpG were determined with Nanodrop. The results showed that when the theoretical drug-loading rate was 10 wt. %, the entrapment rate of the obtained vesicles was 100%. The particle size of the vesicles obtained above was about 50 nm with a narrow distribution.

When TMC was replaced respectively with caprolactone (8-CL), lactide (LA), glycolide (GA), and a 2,4,6-trimethoxy phenyl acetal pentaerythritol carbonate (TMBPEC) monomer, ApoE targeting drug-loaded cross-linked vesicles were obtained according to the above method with an entrapment rate of 98%, 85%, 93% and 86%, respectively.

When CpG ODN 1826 was replaced with CpG ODN 2395 or CpG ODN 2006 and the rest remained unchanged, ApoE targeting drug-loaded cross-linked vesicles were obtained according to the above method with an entrapment rate of 100%.

When the above theoretical drug-loading rate was changed to 5 wt. % or 15 wt. % and the rest remained unchanged, an ApoE targeting drug-loaded cross-linked vesicle was obtained. The drug-loading rate and entrapment rate of CpG were determined with Nanodrop. The results showed that when the theoretical drug-loading rate was 5 wt. % or 15 wt. %, the entrapment rate was 100%, that is, the theoretical drug-loading rate was consistent with the actual drug-loading rate. The particle size of the obtained vesicles was about 50-65 nm with a narrow distribution.

When the molar ratio of MeO-PEG-P(TMC-DTC)-PEI and ApoE-PEG-P(TMC-DTC) was changed and the rest remained unchanged, the drug-loaded cross-linked vesicles with different ApoE targeting densities (5% ApoE targeting group, 15% ApoE targeting group, 20% ApoE targeting group, 25% ApoE targeting group, 30% ApoE targeting group, and 35% ApoE targeting group) were obtained. The drug-loading rate and entrapment rate of CpG were determined with Nanodrop. The results showed that when the theoretical drug-loading rate was 5 wt. %, 10 wt. % and 15 wt. %, the entrapment rate of the targeting drug-loaded vesicles with the ApoE targeting density of 5%, 15% and 20% was 100%, that is, the theoretical drug-loading rate was consistent with the actual drug-loading rate; and the entrapment rate of the targeting drug-loaded vesicles with the ApoE targeting density of 25%, 30% and 35% decreased in turn, which was 75%-90%. The particle size of all the vesicles was 50-85 nm with a narrow distribution.

PS-PEI-CpG loaded with CpG was prepared by a solvent exchange method. The specific steps were as follows: adding a certain amount of CpG (with a theoretical drug-loading rate of 5 wt. % and 10 wt. %, respectively) to 950 μL of a HEPES buffer solution (5 mM, pH 6.8), and then adding 50 μL of a DMSO solution of MEO-PEG-P(TMC-DTC)-PEI (at a polymer concentration of 40 mg/mL) to HEPES and stirring for 10 min; and dialyzing the obtained dispersion in HEPES for 2 h (MWCO 350 kDa), in a mixed buffer solution of HEPES and PB (10 mM, pH 7.4) (v/v, 1/1) for 1 h, and in a PB buffer solution for 2 h to obtain a targeting drug-loaded vesicle, which was recorded as PS-PEI-CpG (with a drug-loading rate of 10 wt. %). The drug-loading rate and entrapment rate of CpG were determined with Nanodrop. The results showed that when the theoretical drug-loading rate was 5 wt. %, 10 wt. % and 15 wt. %, the entrapment rate was 100%, that is, the theoretical drug-loading rate was consistent with the actual drug-loading rate. The particle size of the vesicles obtained above was 50-60 nm with a narrow distribution.

When the drug CpG was replaced with Cy5-labeled granzyme B (GrB), the GrB-loaded vesicles with different ApoE targeting densities were obtained according to the preparation method in Example 6, and were used in Example 8.

When CpG was replaced with GrB and the rest remained unchanged, ApoE-PS-Sp-GrB was obtained according to the method for preparing ApoE-PS-Sp-CpG in Example 6. It was found that when the theoretical drug-loading rate was 5%, the highest entrapment rate of ApoE-PS-Sp-GrB with different grafting densities was 85%; and the particle size was 50-68 nm with a narrow distribution.

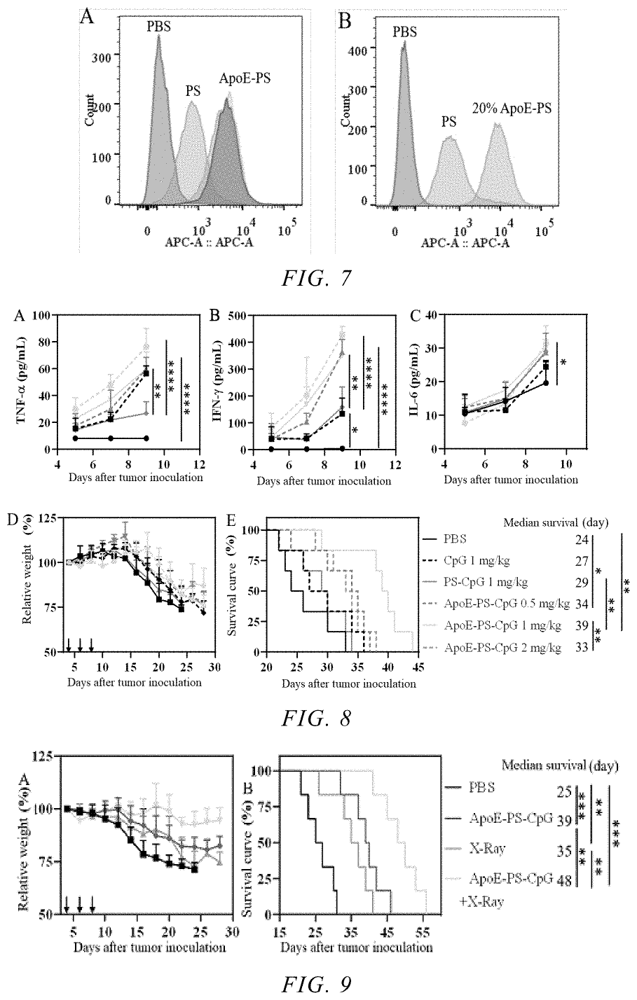

Example 8: Cell endocytosis experiment and simulated penetration of blood brain barrier (BBB) of targeting drug-loaded vesicles: For the cell endocytosis experiment of targeting drug-loaded vesicles, Cy5-labeled granzyme B (GrB) and the vesicles ApoE-PS with different ApoE densities on the surface were taken as an example, and a flow cytometer (FACS) was used for follow-up determination. The steps were as follows: placing 900 μL of a suspension of the 1640 medium of LCPN cells (containing 10% bovine serum, 100 IU/mL penicillin, and 100 IU/mL streptomycin) on a 6-well culture plate (1.5×10⁵ cells per well), and culturing at 37° C. in 5% carbon dioxide for 24 h; adding 100 μL of a PBS solution of Cy5-GrB-loaded vesicles with different ApoE targeting densities to the well (the final concentration of Cy5 was 2 nM), and continuing the incubation for 4 h; and removing the medium, digesting with trypsin (0.25% (w/v), containing 0.03% (w/v) EDTA), and washing twice with PBS. Finally, FACS (BD FACS) was used for the test. The results were shown in FIG. 7A, which indicated that the targeting vesicles ApoE PS could be endocytosed into the LCPN cells more than the no-target PS, and the Cy5 fluorescence values of 10%, 20% and 30% ApoE targeting groups were 4.6, 5.8 and 5.4 times of that of the no-target group, respectively.

In addition, bEnd 3 was used to establish an in-vitro BBB model, so as to investigate the ability of the ApoE vesicles to penetrate BBB. bEnd. 3 was cultured with a DMEM medium (containing 100 U/mL penicillin, 100 U/mL streptomycin, and 10% (v/v) fetal bovine serum) at 37° C. in 5% CO₂. The method for establishing the in-vitro BBB model was as follows: adding a cell culture chamber on a 24-well plate (with an average well diameter of 1.0 μm and a bottom surface area of 0.33 cm²), then adding 800 μL and 300 μL of the DMEM medium to the 24-well plate and the chamber, respectively, and finally inoculating the chamber with 10⁵ cells per well. The integrity of the bEnd. 3 cell monolayer was detected by a microscope and a transmembrane resistance meter, the results showing that there was no gap in the cell monolayer; and the in-vitro BBB model with the transmembrane resistance higher than 200 Ω·cm² was used to investigate the ability of ApoE-PS to penetrate the in-vitro BBB. The steps of research on the penetration of BBB were as follows: adding the Cy5-labeled ApoE-PS samples with different ApoE densities to the chamber (with a polymer concentration of 0.1 mg/mL); and incubating for 24 h, digesting with trypsin (0.25% (w/v), containing 0.03% (w/v) EDTA), and washing twice with PBS. Cy5 fluorescence of each sample was measured by a fluorescence spectrometer. The results showed that the targeting vesicles ApoE-PS could penetrate the BBB model more than the no-target PS. FIG. 7B showed that the Cy5 fluorescence value of the 20% ApoE targeting group was 11.6 times that of the no-target group.

Example 9: Therapeutic effects of different CpG formulations and different dosages on in-situ mouse brain glioma LCPN model mice studied by caudal vein administration: The establishment of in-situ mouse brain glioma LCPN model mice was as follows: selecting C57BL/6J mice weighing about 18-20 g and aged 6-8 weeks, using a No. 26 Hamilton syringe to inject 5 μL containing 5×10⁴ LCPN cells into the right skull (+1.0 mm anterior, 2.5 mm lateral, and 3.0 mm deep) through a brain stereotaxic instrument, and retaining for 5 min; in the 4th day after the inoculation, randomly dividing the mice into 6 groups (6 mice in each group), i.e. PBS, free CpG (1 mg/kg), PS-Sp-CpG (1 mg/kg), and ApoE-PS-Sp-CpG (0.5 mg/kg, 1 mg/kg, and 2 mg/kg); on the 4th, 6th and 8th day after the inoculation, injecting each drug into the mice through the caudal vein, and on the 5th, 7th and 9th day after the inoculation, taking blood from the eye socket to monitor changes of the concentrations of TNF-α, IFN-γ and IL-6 in the mouse plasma; and weighing the mice every two days during the 4th to 28th days. In FIG. 8, A, B and C represented the changes of the concentrations of TNF-α, IFN-γ and IL-6 in the plasma of the mice in each group. It could be seen from the figure that each CpG treatment group could significantly increase the concentrations of the three cytokines in the mouse plasma, with the ApoE targeting group having the most obvious effect. D represented the weight change of mice in each group, and E represented the survival curve. It could be seen from the figure that the ApoE targeting treatment group could delay the trend of weight loss in mice, and the dosage of 1 mg/kg could achieve the best therapeutic effect; compared with the PBS group, free CpG group and PS-CpG group, the survival period of mice could be significantly prolonged (39 vs. 24, 27 and 29 days, **p).

Example 10: Therapeutic effects of ApoE-PS-Sp-CpG combined with radiotherapy (X-Ray) on in-situ mouse brain glioma LCPN model mice studied by caudal vein administration: The in-situ mouse brain glioma LCPN model mice were established as per Example 9, with the steps as follows: in the 4th day after the inoculation, randomly dividing the mice into 4 groups (6 mice in each group), i.e. PBS, X-Ray (3Gy/time), ApoE-PS-Sp-CpG (1 mg/kg), and ApoE-PS-Sp-CpG (1 mg/kg)+X-Ray (3Gy/time); on the 4th, 6th and 8th day after the inoculation, injecting ApoE-PS-Sp-CpG into the mice via the caudal vein, and after 6 h irradiating the mice with X-Ray; and weighing the mice every two days during the 4th to 28th days. In FIG. 9, A represented the weight change of mice, and B represented the survival curve. Compared with the PBS group, X-Ray and ApoE-PS-Sp-CpG alone or in combination could delay the weight loss and prolong the survival period of mice, with the combination group having the most obvious effects (having the smallest weight loss and the longest survival period (25, 35, 39 and 48 days).

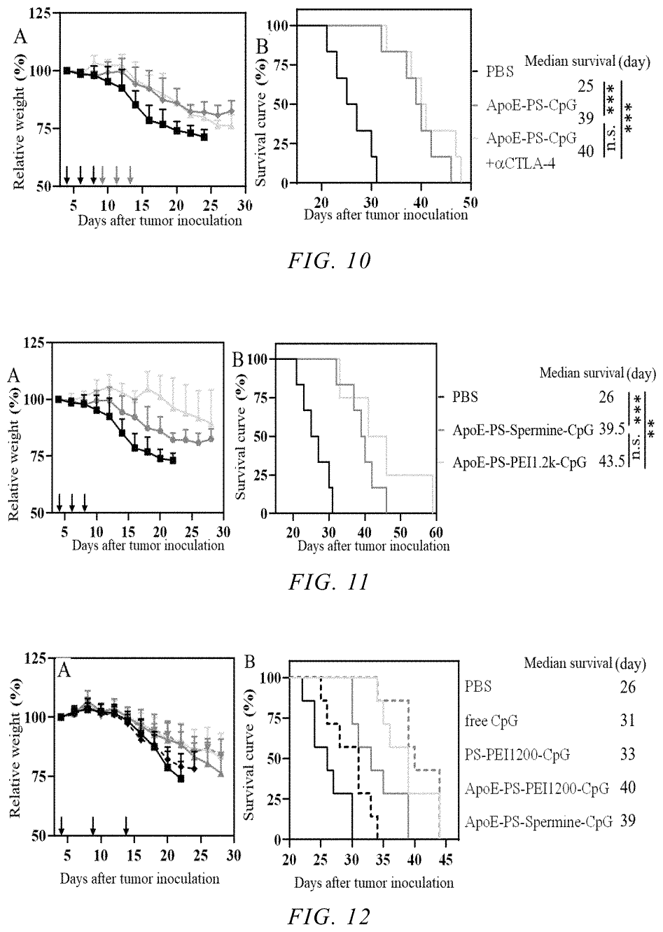

Example 11: Therapeutic effects of ApoE-PS-Sp-CpG combined with αCTLA-4 antibody on in-situ mouse cerebral glioma LCPN model mice studied by caudal vein administration: The in-situ mouse brain glioma LCPN model mice were established as per Example 9, with the steps as follows: in the 4th day after the inoculation, randomly dividing the mice into 3 groups (6 mice in each group), i.e. PBS, ApoE-PS-Sp-CpG (1 mg/kg), and ApoE-PS-Sp-CpG (1 mg/kg)+αCTLA-4 (10 mg/kg); on the 4th, 6th and 8th day after the inoculation, injecting ApoE-PS-Sp-CpG into the mice of the two later groups through the caudal vein, and on the 9th, 11th and 13th day after the inoculation, injecting αCTLA-4 into the mice of the third group through the intraperitoneal administration; and weighing the mice every two days during the 4th to 28th days. In FIG. 10, A represented the weight change of mice of each group, and B represented the survival curve. Compared with the PBS group, ApoE-PS-Sp-CpG (1 mg/kg) could significantly delay the trend of weight loss and prolong the survival period of mice, but the combination of a CTLA-4 did not further enhance the therapeutic effects (the survival period was 25, 39 and 40 days, respectively, ***p).

Example 12: Therapeutic effects of ApoE-PS-Sp-CpG and ApoE-PS-PEI1.2k-CpG on in-situ mouse cerebral glioma LCPN model mice compared by caudal vein administration: The in-situ mouse brain glioma LCPN model mice were established as per Example 9, with the steps as follows: in the 4th day after the inoculation, randomly dividing the mice into 3 groups (6 mice in each group), i.e. PBS, ApoE-PS-Sp-CpG (1 mg/kg), and ApoE-PS-PEI1.2k-CpG (1 mg/kg); on the 4th, 6th and 8th day after the inoculation, injecting the drug into the mice through the caudal vein; and weighing the mice every two days during the 4th to 28th days. In FIG. 11, A represented the weight change of mice of each group, and B represented the survival curve. Compared with the PBS group, both the ApoE-PS-Sp-CpG group and the ApoE-PS-PEI1.2k-CpG group could significantly delay the trend of weight loss and prolong the survival period of mice (***p), and the therapeutic effect of the ApoE-PS-PEI1.2k-CpG group was slightly better than that of the ApoE-PS-Sp-CpG group (26, 39.5 and 43.5 days), indicating that the positively charged substance in the inner shell of vesicle formed by a polymer had an impact on the therapeutic effect.

Example 13: Therapeutic effects of different CpG formulations on in-situ mouse cerebral glioma LCPN model mice studied by nasal vein administration: The in-situ mouse brain glioma LCPN model mice were established as per Example 9, with the steps as follows: in the 4th day after the inoculation, randomly dividing the mice into 5 groups (7 mice in each group), i.e. PBS, free CpG (0.5 mg/kg), PS-PEI1.2k-CpG (0.5 mg/kg), ApoE-PS-PEI1.2k-CpG (0.5 mg/kg), and ApoE-PS-Sp-CpG (0.5 mg/kg); on the 4th, 9th and 14th day after the inoculation, injecting the drug into the mice through the nasal vein; and weighing the mice every two days during the 4th to 28th days. In FIG. 12, A represented the weight change of mice of each group, and B represented the survival curve. and compared with the PBS group, the CpG group and the PS-PEI1.2k-CpG group, the ApoE-PS-PEI1.2k-CpG group could significantly prolong the survival period of mice (26, 31, 33 and 40 days).

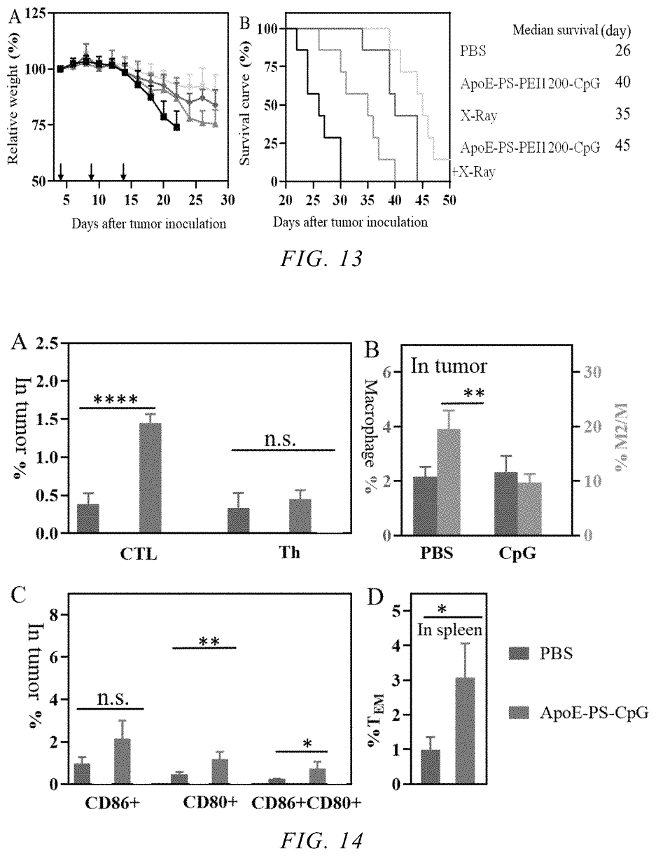

Example 14: Therapeutic effects of ApoE-PS-PEI1.2k-CpG combined with radiotherapy on in-situ mouse cerebral glioma LCPN model mice studied by nasal vein administration: The in-situ mouse brain glioma LCPN model mice were established as per Example 9, with the steps as follows: in the 4th day after the inoculation, randomly dividing the mice into 4 groups (7 mice in each group), i.e. PBS, X-Ray (3Gy/time), ApoE-PS-PEI1.2k-CpG (0.5 mg/kg), and ApoE-PS-PEI1.2k-CpG (0.5 mg/kg)+X-Ray (3Gy/time); on the 4th, 9th and 14th day after the inoculation, first irradiating the mice with X-Ray, and 6 h after the irradiation, injecting ApoE-PS-PEI1.2k-CpG into the mice through the nasal vein; and weighing the mice every two days during the 4th to 28th days. In FIG. 13, A represented the weight change of mice, and B represented the survival curve. Compared with the PBS group, X-Ray and ApoE-PS-Sp-CpG (0.5 mg/kg) alone or in combination could delay the trend of weight loss and prolong the survival period of mice, with the combination group having the most obvious effects (26, 35, 40 and 45 days).

Example 15: Analysis of immune cells in tumor and spleen of mice bearing in-situ LCPN: Conventional methods were used to analyze the immune cells in the tumor and spleen of mice bearing in-situ LCPN (n=3, Example 9). The results were shown in FIG. 14, where A represented the percentage of CTL (CD8+ T cells) and Th (CD4+ T cells) in the tumor, B represented the percentage of macrophages (CD11b+F4/80+) and M2 phenotype (CD11b+F4/80+CD206+) in the tumor, C represented the percentage of activated CD86+ and/or CD80+ APC in the tumor, and D represented the percentage of effector memory T cells (CD8+CD44+CD62L−) in the spleen. These data indicated that ApoE-PS-CpG could trigger the innate and adaptive immune response in the tumor microenvironment by activating CTL, significantly recruit tumor antigen presenting cells APC, reduce M2 phenotype macrophages and stimulate macrophages, and produce certain immune memory effects.

A MTT method was as follows: inoculating human breast cancer cells (MCF-7) in a 96-well plate at 5×10³ cells/mL, 80 μL per well, and culturing the cells for over 24 h until the cells adhered to the wall by about 70%; preparing the vesicles formed by a cross-linked polymer according to Examples 6 and 7, without adding drugs; then adding the vesicles with different concentrations (0.1-0.5 mg/mL) to each well of the experimental group, and providing a cell blank control well and a culture-medium blank well (multiple 4 wells); after 24 h of incubation, adding 10 μL of MTT (5.0 mg/mL) to each well; and continuing the culture for 4 h, and then adding 150 μL of DMSO to each well to dissolve the generated crystallite. A microplate reader was used to measure the absorbance value at 492 nm, with the zeroing carried out according to the culture-medium blank well, so as to calculate the survival rate of cells. The results showed that when the concentrations of various vesicles formed by a cross-linked polymer (targeting, non-targeting, and different hydrophobic chain segments) increased from 0.1 mg/mL to 0.5 mg/mL, the survival rate of MCF-7 was still higher than 88%, indicating that the vesicles formed by a cross-linked polymer of the present invention had good biocompatibility.

The test objects were ApoE-PS-Sp-CpG in Example 6, and ApoE-PS-PEI-CpG in Example 7. The toxicity of drug-loaded vesicles to MCF-7 cells was studied. The concentration of CpG was 0.05 mg/mL, and the free CpG was used as a control. Culture of cells was the same as above. After 4 h of co-culture, the sample was drawn out and replaced with a fresh medium for further incubation for 68 h. The subsequent MTT addition, treatment and absorbance determination were the same as those in the above examples. The results showed that the survival rates of the MCF-7 cells treated with the targeting vesicle formed by a cross-linked polymer ApoE-PS-Sp-CpG and ApoE-PS-PEI-CpG and free CpG were about 85%, 91% and 97%, respectively.

A test of toxicity of the above drug-loaded vesicles formed by a polymer to the LCPN cells was also conducted, with the experimental operation the same as above. The results showed that the survival rates of the LCPN cells treated with the targeting vesicle formed by a cross-linked polymer ApoE-PS-Sp-CpG and ApoE-PS-PEI-CpG and free CpG were about 90%, 82% and 98%, respectively.

Animal selection was the same as that in Example 12. The steps were as follows: injecting 1×10⁷ MCF-7 cells subcutaneously; starting the experiment about 3.5 weeks later when the tumor size was 100 mm³; randomly dividing the mice into 3 groups (6 mice in each group), i.e. PBS, ApoE-PS-Sp-CpG (1 mg/kg), and ApoE-PS-PEI1.2k-CpG (1 mg/kg); on the 4th, 6th and 8th day after the inoculation, injecting the drug into the mice through the caudal vein; and weighing the mice every two days during the 0th to 28th days. The median survival period of the PBS group, the ApoE-PS-PEI1.2k-CpG group and the ApoE-PS-Sp-CpG group was 29, 30.5 and 31 days, respectively (the subcutaneous tumor was judged dead when it grew to 1000 mm³).

In the following example, ApoE-PS-Sp-CpG in Example 6 was used as ApoE-PS-CpG. Correspondingly, the targeting ApoE was removed to obtain PS-CpG. Cy3 could be routinely marked on CpG according to experimental needs.

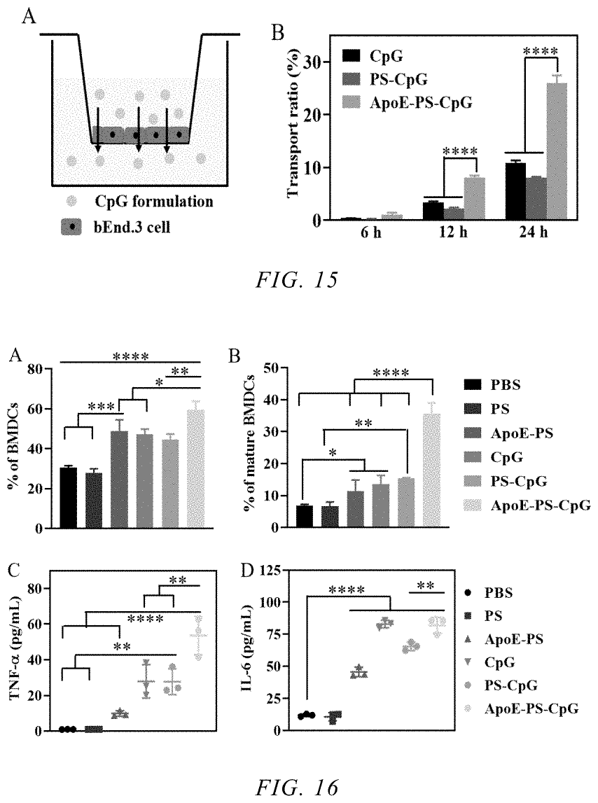

Example 16: In-vitro simulation of ApoE-PS-CpG penetrating BBB: Taking the vesicle ApoE-PS-CpG loaded with CpG labeled with Cy3 (CpG-Cy3) as an example, the in-vitro BBB model was established according to the method of Example 8. FIG. 15A showed a schematic diagram of the established in-vitro BBB model. The steps of research on the penetration of BBB were as follows: adding samples of CpG-Cy3, PS-vCpG-Cy3 and ApoE-PS-CpG-Cy3 to the chamber (calculated based on CpG-Cy3, 1 μg/well) (n=3); and in 6, 12 and 24 h, collecting all the culture media in the lower layer respectively, and adding 800 μL of fresh DMEM culture medium as a supplement. Cy3 fluorescence of each sample was measured by a fluorescence spectrometer. The penetration efficiency was defined as the cumulative amount of CpG-Cy3 penetrating BBB/the initial amount of CpG-Cy3 added. FIG. 15B showed that the ApoE targeting group had higher penetration efficiency than the free CpG group and the no-target group.

Example 17: Experiment of ApoE-PS-CpG activating BMDC in vitro: According to the conventional method, immune cells were extracted from the bone marrow of C5BL/6J mice and induced to differentiate into immature BMDC in vitro with GM-CSF (20 ng/mL); and the activation of immature BMDC by empty carriers (PS, ApoE-PS, with a polymer concentration of 4 μg/mL) and different CpG formulations (CpG, PS-CpG, ApoE-PS-CpG, with a CpG concentration of 0.4 μg/mL and a polymer concentration of 4 μg/mL) was studied. The results showed that both ApoE-PS and the CpG formulations could increase the proportion of DC cells (CD11c⁺) (FIG. 16A), indicating that these samples could promote the transformation of monocytes into DC cells, but only ApoE-PS-CpG could significantly promote the maturation of DC cells (CD80⁺CD86⁺/CD11c⁺>50%) (FIG. 16B). In addition, by testing the cytokines in the culture media of different groups of cells, it was found that the ApoE-PS-CpG group could significantly increase the expression level of TNF-α (FIG. 16C) and TL-6 (FIG. 16D) secreted by cells compared with other groups.

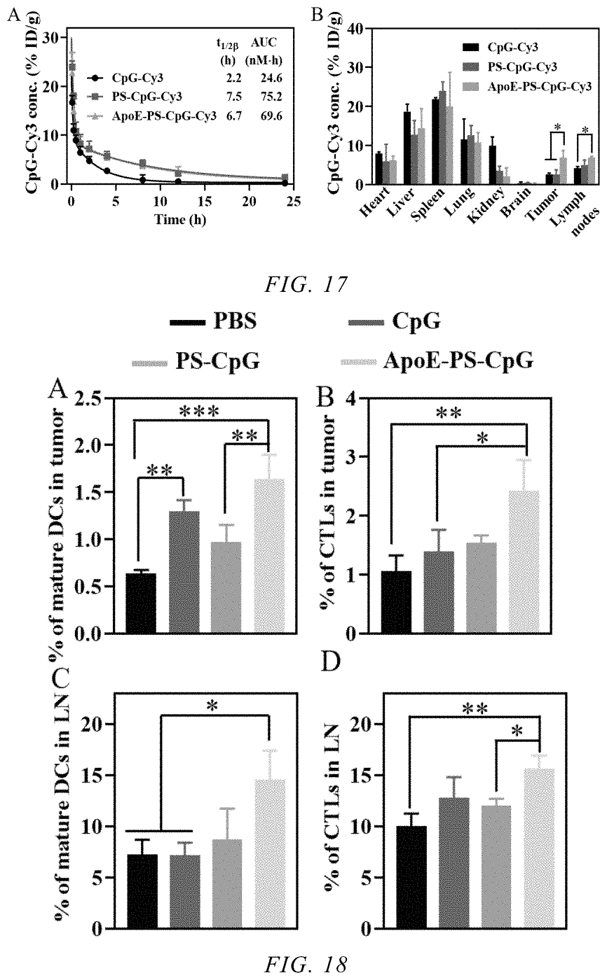

Example 18: Experiments of in-vivo pharmacokinetics of different CpG formulations and biological distribution of main organs: C57BL/6J mice weighing 18-20 g and aged 6-8 weeks were selected for the experiment. CpG-Cy3 with a fluorescent label and CpG without a fluorescent label (m/m 1/3) were used to conduct the in-vivo pharmacokinetics and biological distribution experiments. The total dose of CpG was 1 mg/kg. The pharmacokinetic experiments were carried out in healthy mice, with the steps as follows: injecting different CpG formulations into the caudal vein of mice, and then taking about 70 μL of whole blood from the eye socket at a set time point; and immediately adding the blood to an EP tube pretreated with heparin sodium, and centrifugating to obtain 20 μL of plasma; damaging the plasma with 600 μL of DMSO (including 20 mM DTT), and detecting with a fluorescence spectrometer. The results showed that the CpG nano adjuvant loaded on the vesicle formed by a polymer could significantly prolong the half-life of CpG (7.5, 6.7 vs. 2.2 h) and AUC (75.2, 69.6 vs. 24.6 nM h) compared with free CpG (FIG. 17A). The biological distribution experiment was carried out with the in-situ LCPN model mice, which were divided into 3 groups, with 3 mice in each group. The steps were as follows: using a No. 26 Hamilton syringe to inject 5 μL containing 5×10⁴ LCPN cells into the right skull (+1.0 mm anterior, 2.5 mm lateral, and 3.0 mm deep) through a brain stereotaxic instrument, and retaining for 5 min; in the 9th day after the inoculation, randomly dividing the mice into 3 groups (3 mice in each group); and in 12 h after caudal vein administration, dissecting various organs of mice and quantifying CpG-Cy3 by a fluorescence spectrometer. The results showed that, compared with the free and no-target groups, the mice in the ApoE targeting group had high CpG-Cy3 enrichment in brain tumors and cervical lymph nodes (FIG. 17B).

Example 19: Flow analysis experiments of different CpG formulations activating tumors and immune cells in lymph nodes in vivo: The steps were as follows: selecting C57BL/6J mice weighing about 18-20 g and aged 6-8 weeks, using a No. 26 Hamilton syringe to inject 5 μL containing 5×10⁴ LCPN cells into the right skull (+1.0 mm anterior, 2.5 mm lateral, and 3.0 mm deep) through a brain stereotaxic instrument, and retaining for 5 min; in the 4th day after the inoculation, randomly dividing the mice into 4 groups (3 mice in each group), i.e. PBS, free CpG (1 mg/kg), PS-Spermine-CpG (1 mg/kg), and ApoE-PS-Spermine-CpG (1 mg/kg); on the 4th, 6th and 8th day after the inoculation, injecting the drug into the mice through the caudal vein; and dissecting the brain tumors and cervical lymph nodes of mice on the day (D9) after all the drugs were administered, staining DC cells with CD11c, CD80 and CD86, and staining T cells with CD4 and CD8. A, B, C and D in FIG. 18 represented the proportion of mature DC (CD11c⁺CD80⁺CD86⁺) and CTL (CD8⁺) in the tumors of mice in each group, and the proportion of mature DC and CTL in the cervical lymph nodes, respectively. The results showed that the proportion of mature DC and CTL in the brain tumors and lymph nodes of mice in the ApoE targeting group was higher than that in other groups.

In theory, CpG, as a TLR activator, can induce an anti-tumor immune response of cells. However, it was found in the early clinical follow-up visit of glioma and melanoma patients by the existing technology that the application results were not optimistic, mainly because CpG caused an inflammatory reaction and brain edema. In order to meet the requirement that CpG, as a small molecule immune adjuvant, needs to enter the antigen presenting cell APC to play a role, the existing technology adopts the method of intracranial administration, which inevitably has many defects. The loaded adjuvant CpG based on a vesicle formed by a cross-linked biodegradable polymer first disclosed by the present invention achieves an entrapment rate of 100%; it can be injected through the caudal vein or nasal vein as a separate nano vaccine or nano immune adjuvant for efficient immunotherapy of tumors, in particular solving the technical bias of the prior art that CpG needs to be administered intracranially. The experiments prove that the administration of the nano adjuvant of the present invention can avoid immunotoxicity and greatly prolong the survival period of mice.

1. An anti-tumor nano adjuvant based on a vesicle formed by a cross-linked biodegradable polymer, characterized in that: the adjuvant is obtained by loading a drug on the vesicle formed by a reversibly cross-linked biodegradable polymer with an asymmetric membrane structure; the drug is an oligonucleotide that can activate an immune response; the vesicle formed by a reversibly cross-linked biodegradable polymer with an asymmetric membrane structure is obtained by means of the self-assembly of a polymer, or the self-assembly of a polymer and a targeting polymer; the polymer includes a hydrophilic chain segment, a hydrophobic chain segment and positively charged molecules; the targeting polymer includes a targeting molecule, a hydrophilic chain segment and a hydrophobic chain segment; and the hydrophobic chain segment is a polycarbonate chain segment and/or a polyester chain segment.

2. The anti-tumor nano adjuvant based on a vesicle formed by a cross-linked biodegradable polymer according to claim 1, characterized in that: the hydrophilic chain segment is polyethylene glycol; the hydrophobic chain segment contains a disulfide five-membered cyclic carbonate unit; the positively charged molecules include spermine and polyethyleneimine; and the molecular weight of the hydrophobic chain segment is 1.5-5 times that of the hydrophilic chain segment, and the molecular weight of the positively charged molecule is 2%-40% of that of the hydrophilic chain segment.

3. The anti-tumor nano adjuvant based on a vesicle formed by a cross-linked biodegradable polymer according to claim 2, characterized in that: the molecular weight of polyethylene glycol is 5000-7500 Da; the molecular weight of polyethyleneimine is 7%-40% of that of polyethylene glycol; and the molecular weight of spermine is 2.7%-4% of that of polyethylene glycol.

4. The anti-tumor nano adjuvant based on a vesicle formed by a cross-linked biodegradable polymer according to claim 1, characterized in that: the oligonucleotide that can activate an immune response is CpG; and the targeting molecule is ApoE polypeptide.

5. The anti-tumor nano adjuvant based on a vesicle formed by a cross-linked biodegradable polymer according to claim 1, characterized in that: the chemical structural formula of the polymer is as follows: 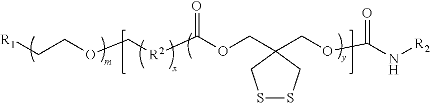
and the chemical structural formula of the targeting polymer is as follows: 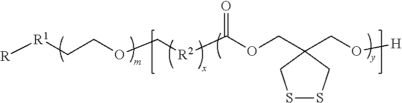
where R₁ is an end group of the hydrophilic chain segment; R₂ is a positively charged molecule; R is a targeting molecule; R¹ is a targeting molecule linkage group; and R² is a cyclic ester monomer, or a unit of a cyclic carbonate monomer after ring opening.

6. A preparation method for the anti-tumor nano adjuvant based on a vesicle formed by a cross-linked biodegradable polymer according to claim 1, characterized in that the method comprises the following steps: preparing the anti-tumor nano adjuvant based on a vesicle formed by a cross-linked biodegradable polymer by a solvent displacement method using a polymer and an oligonucleotide that can activate an immune response as raw materials; or preparing the anti-tumor nano adjuvant based on a vesicle formed by a cross-linked biodegradable polymer by a solvent displacement method using a polymer, a targeting polymer, and an oligonucleotide that can activate an immune response as raw materials.

7. The preparation method for the anti-tumor nano adjuvant based on a vesicle formed by a cross-linked biodegradable polymer according to claim 6, characterized in that: the oligonucleotide that can activate an immune response is CpG; and the targeting molecule is ApoE polypeptide.

8. Use of the anti-tumor nano adjuvant based on a vesicle formed by a cross-linked biodegradable polymer according to claim 1 in preparing an anti-tumor drug.

9. The use according to claim 8, characterized in that the anti-tumor drug is an anti-brain tumor drug.

10. Use of the vesicle formed by a reversibly cross-linked biodegradable polymer with an asymmetric membrane structure as a carrier of the oligonucleotide that can activate an immune response, or use of the vesicle in preparing a carrier of the oligonucleotide that can activate an immune response; wherein the vesicle formed by a reversibly cross-linked biodegradable polymer with an asymmetric membrane structure is obtained by means of the self-assembly of a polymer, or the self-assembly of a polymer and a targeting polymer; the polymer includes a hydrophilic chain segment, a hydrophobic chain segment and positively charged molecules; the targeting polymer includes a targeting molecule, a hydrophilic chain segment and a hydrophobic chain segment; and the hydrophobic chain segment is a polycarbonate chain segment and/or a polyester chain segment.
